(19) United States
(12) Patent Application Publication
(10) Pub. No.: US 2009/0156496 A1
(43) Pub. Date: Jun. 18, 2009

(54) METHODS AND COMPOSITIONS FOR TREATING AND PREVENTING PERIPHERAL NERVE DAMAGE

(75) Inventors: Larry I. Benowitz, Newton, MA (US); Yuqin Yin, Brighton, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, INC., Boston, MA (US)

(21) Appl. No.: 12/300,591

(22) PCT Filed: May 14, 2007
(86) PCT No.: PCT/US2007/011576

§ 371 Date: Nov. 12, 2008

Related U.S. Application Data
(60) Provisional application No. 60/800,068, filed on May 12, 2006.

(51) Int. Cl. A61K 38/17

(52) U.S. Cl. 514/12

(57) ABSTRACT

Disclosed herein is a method for treating and/or preventing peripheral nerve damage in a subject comprising administering to the subject a therapeutically effective amount of oncomodulin. Preferably, the subject is a mammal, most preferably, a human. In preferred embodiments, the oncomodulin may be used in combination with mannose, a mannose derivative and/or inosine.

### RELATED APPLICATIONS

This application is an International Application, which claims the benefit of priority under 35 U.S.C. §119(e) of U.S. Provisional Application Ser. No. 60/800,068 filed on May 12, 2006, the contents of which are incorporated herein by reference in its entirety.

### GOVERNMENT SUPPORT

This invention was supported, in part, by National Institutes of Health (NIH) Grant No. EY 05690. The government of the United States may have certain rights to the invention.

### BACKGROUND OF THE INVENTION

Peripheral neuropathy describes damage to the peripheral nervous system. It can manifest itself as a dysfunction of motor, sensory, sensorimotor or autonomic nerves.

The disorder is associated with a wide variety of causes, including genetically acquired conditions, systemic disease or exposure to toxic agents. Diabetic neuropathy is one example of disease-induced peripheral neuropathy. Neuropathies can also occur in conditions such as acromegaly, hypothyroidism, AIDS, leprosy, Lyme disease, systemic lupus erythematosus, rheumatoid arthritis, Sjogren's syndrome, periarteritis nodosa, Wegener's granulomatosis, cranial arteritis, and sarcoidosis, as well as other conditions.

There is a strong need in the art for treatments of peripheral nerve damage (peripheral neuropathy).

### SUMMARY OF INVENTION

The present invention provides a method for treating and/or preventing peripheral nerve damage in a subject comprising selecting a subject having peripheral nerve damage or in need of prevention of such damage, and administering to the subject a therapeutically effective amount of oncomodulin. Preferably, the subject is a mammal, most preferably, a human.

In one embodiment, a cAMP modulator and/or an axogenic factor is further administered to the subject. The components can be used separately, but administered contemporaneously. While not wishing to be bound by a particular theory, it is believed that the cAMP modulator and axogenic factor potentiates the activity of the oncomodulin.

Preferably, the cAMP modulator is non-hydrolyzable cAMP analogues, forskolin, adenylate cyclase activators, macrophage-derived factors that stimulate cAMP, macrophage activators, calcium ionophores, membrane depolarization, phosphodiesterase inhibitors, specific phosphodiesterase IV inhibitors, beta2-adrenoreceptor inhibitors or vasoactive intestinal peptide.

Preferred axogenic factors include mannose (sometimes referred to as “AF-1”), mannose derivatives and inosine.

The compositions may be administered systemically or locally such that the composition is brought into contact with peripheral neurons of the subject.

Aspects of the present invention relate to a method for treating and/or preventing peripheral nerve damage in a subject comprising administering to the subject a therapeutically effective amount of oncomodulin, to thereby treat and/or prevent peripheral nerve damage in the subject. The peripheral nerve damage may be in the subject's spinal cord. Another aspect of the present invention relates to a method for treating and/or preventing spinal cord injury in a subject comprising administering to the subject a therapeutically effective amount of oncomodulin to thereby treat and/or prevent spinal cord injury in the subject. These methods may optionally further comprise a step of selecting a subject in need of treatment or prevention of such peripheral nerve damage. In one embodiment, the methods further comprise administering to said subject a cAMP modulator. The cAMP modulator can be non-hydrolyzable cAMP analogues, forskolin, adenylate cyclase activators, macrophage-derived factors that stimulate cAMP, macrophage activators, calcium ionophores, membrane depolarization, phosphodiesterase inhibitors, specific phosphodiesterase IV inhibitors, beta2-adrenoreceptor inhibitors or vasoactive intestinal peptide, or combinations thereof. In one embodiment, the methods further comprise administering mannose or a mannose derivative to said subject. In one embodiment, the methods further comprise administering inosine to said subject. The peripheral nerve damage can be the result of diabetic neuropathy, of a viral or bacterial infection. The oncomodulin may be administered topically, by local injection. The oncomodulin can be administered to the subject in a pharmaceutically acceptable formulation. The subject of the method may be a mammal, e.g. a human.

Another aspect of the present invention relates to an article of manufacture comprising packaging material and a pharmaceutical agent contained within said packaging material, wherein said packaging material comprises a label which indicates said pharmaceutical may be administered, for a sufficient term at an effective dose, for treating and/or preventing peripheral nerve damage together with a pharmaceutically acceptable carrier, wherein the pharmaceutical agent comprises oncomodulin.

Another aspect of the present invention relates to a pharmaceutical kit for the treatment and/or prevention of damage to peripheral nerves comprising the combination of oncomodulin, an axogenic factor, and a cAMP modulator. The axogenic factor can be mannose, a mannose derivative or inosine. Examples of cAMP modulators are non hydrolyzable cAMP analogues, forskolin, adenylate cyclase activators, macrophage-derived factors that stimulate cAMP, macrophage activators, calcium ionophores, membrane depolarization, phosphodiesterase inhibitors, specific phosphodiesterase IV inhibitors, beta2-adrenoreceptor inhibitors or vasoactive intestinal peptide.

Another aspect of the present invention relates to the use of oncomodulin in the preparation of a medicament for treating and/or preventing peripheral nerve damage in a subject. This use is envisioned as described in the methods herein.

Another aspect of the present invention relates to a method for inhibiting the axogenic effects of oncomodulin on a neuron comprising contacting an inhibitor of oncomodulin to the neuron. In one embodiment, the neuron is in a subject in need of inhibition of oncomodulin axogenic effects, and contacting is achieved by administering the inhibitor to the subject.

### BRIEF DESCRIPTION OF FIGURES

FIG. 1: Oncomodulin stimulates axon regeneration in RGCs. FIG. 1A is a schematic of oncomodulin and other related calcium binding proteins. Oncomodulin (OM) contains two active Ca²⁺-binding sites (rectangles) and is related to α-parvalbumin (α-PV), calmodulin (CM), calbindin (CB) and S100-β in its EF-hand domain, but only to α-PV in its N-terminal region (% sequence identity indicated). FIG. 1B is a photomicrograph of variously treated RGCs showing the effect of oncomodulin on RGCs. Top, cells were treated as indicated and stained with an antibody to GAP-43. Bottom, same fields showing Fluorogold labeling to identify RGCs. Scale bar, 30 μm. FIG. 1C is a histogram showing the percentage of RGCs extending axons in the designated size ranges (30-70 μm, 70-140 μm and >140 μm) after 3 d in culture with the indicated factors. Differences between treatment groups are all significant at P<0.0001. FIG. 1D is a bar graph of data indicating axon outgrowth in response to oncomodulin, mannose and forskolin. Histogram data, as shown in FIG. 1C, is collapsed to obtain the total percentage of Fluorogold-labeled RGCs whose axons are >30 μm in length. ***P<0.001, difference from forskolin and mannose alone. FIG. 1E is a bar graph of data indicating cell survival (average number of RGCs per 400× microscope field, normalized to survival in controls treated with defined media alone). FIG. 1F is a line graph of data indicating a dose response of axon outgrowth to the presence of oncomodulin. (^{†}P<0.05, decrease relative to controls). MCM, media containing proteins secreted by zymosan-stimulated macrophages.

FIG. 2 Potency and specificity of oncomodulin. FIG. 2 A, B and C are bar graphs, with FIG. 2 C also containing a photograph above the bar graph. FIG. 2A indicates axon-promoting effects of oncomodulin (OM) versus BDNF (50 ng ml⁻¹), CNTF (10 ng ml⁻¹) and GDNF (50 ng ml⁻¹). Factors were tested in the absence (light bars) or presence (dark bars) of forskolin and mannose; results are quantified as in FIG. 1D., *P<0.05 and **P<0.001, difference from growth induced by forskolin plus mannose. ^{†††}P<0.001, difference in effect of oncomodulin versus CNTF. FIG. 2B indicates axon-promoting effects of oncomodulin and other Ca²⁺-binding proteins, including parvalbumin (PV), calmodulin (CaM), calbindin (CB) and S100-β (S100) (all tested at 15 nM in the presence of forskolin and mannose). FIG. 2C indicates immunodepletion of oncomodulin from MCM (containing proteins secreted by zymosan-stimulated macrophages) eliminated axon-promoting activity. Top photo is of western blotting to detect oncomodulin in MCM after exposure to blank protein A beads (−), or after depletion using anti-oncomodulin IgG (α-OM) or IgG from normal rabbit serum NRS) bound to protein A beads. Bottom, axon-promoting activity of MCM after oncomodulin depletion. ^{††}P<0.01, decrease relative to nondepleted MCM.

FIG. 3 Oncomodulin binding to RGCs: kinetics and domain analysis. RGCs were retrogradely labeled with Fluorogold 7 d before being isolated by immunopanning and grown in culture with or without forskolin. FIGS. 3A-3J are photomicrographs. FIGS. 3A and 3B data indicate the purity of cultures: phase (a) and fluorescent (b) image of Fluorogold-labeled RGCs. Scale bar, 30 μm. FIGS. 3C-3J indicate binding of alkaline phosphatase (AP)-oncomodulin fusion proteins (AO) to RGCs (all at 10 nM): AP alone (c, with forskolin); AO in the absence (FIG. 3D) or presence (FIG. 3E) of forskolin. cAMP-dependent AO binding was displaced by a 100-fold excess of unlabeled oncomodulin (OM, FIG. 3F) but not by α-parvalbumin (FIG. 3G), by AP fusion proteins containing the N terminus (AO^{NT}, FIG. 3H) or C-terminus (AO^{CT}, FIG. 3I) of oncomodulin, and by AO binding to permeabilized RGCs not pretreated with forskolin (FIG. 3J). Scale bar in c-j, 30 μm. FIG. 3K is a bar graph of data which indicates quantitation of binding (absorbance per cell surface area corrected by AP binding. ***P<0.001, increase relative to binding without forskolin. ^{†††}P<0.001, reduction relative to AO plus forskolin). FIG. 3L is a bar graph of data which indicates the effect of AO mutations (AO var) on axon outgrowth (tested at 15 nM with forskolin+mannose (‘Forsk’, ‘M’) present; outgrowth measured as in FIG. 1C). E62N/E101Q is an AO mutant with amino acid substitutions that lower Ca²⁺ binding. ***P<0.001, increase relative to forskolin+mannose. FIG. 3M are also graphs showing binding kinetics and a corresponding Scatchard plot. AO binding to RGCs was saturable (K_{d}˜28±5 nM; i.u., intensity units, corrected by AP binding). FIG. 3N is a line graph of binding data. After equilibrium binding of AO (40 nM), cells were exposed to unlabeled oncomodulin as shown. Results are normalized to AO binding in the absence of competitor.

FIG. 4 The downstream effects of oncomodulin involve CaMKII and transcriptional changes. FIGS. 4 A, B, and C are graphs of data quantitating axon outgrowth in response to the indicated factos. FIG. 4A indicates the effects of oncomodulin (OM), but not of forskolin plus mannose, were blocked by KN93, an inhibitor of CaMKII. KN92 is an inactive form of KN93. Inhibitors of MEK-1, MEK-2 or MEK-5 (PD: PD98059), P13 kinase (LY: LY294002), or Jak-1, Jak-2 or Jak-3 (Jaki) did not block oncomodulin-induced growth, although all three combined (‘L’, ‘P’, ‘J’) blocked outgrowth below the level of forskolin+mannose. FIG. 4B indicates the effects of oncomodulin are blocked by the transcriptional inhibitor ActD. FIG. 4C indicates that elevating [cAMP] did not mimic the effect of oncomodulin. Although cAMP was required for oncomodulin activity (FIG. 1C), increasing [cAMP] beyond an optimal level was deleterious. FIG. 4D indicates that oncomodulin increased levels of P-CREB. Retinas were prepared for histology 2 h after intravitreal injections. Sections were stained with 4′,6-diamidino-2-phenylindole (DAPI) and with antibodies to P-CREB and class III β-tubulin. gcl, ganglion cell layer; ipl, inner plexiform layer. Arrows point to P-CREB-positive RGCs. ***P<0.001, decrease relative to outgrowth in the absence of the inhibitor. Scale bar, 30 μm.

FIG. 5 Oncomodulin expression and secretion. FIG. 5A is a photograph showing vesicular localization of oncomodulin. Shown is a confocal image of cultured macrophage stained with DAPI and an anti-oncomodulin antibody followed by a fluorescent secondary antibody. Scale bar, 5 μm. FIG. 5B is a collection of eight photographs of western blots. The data indicates secretion of oncomodulin (OM). Macrophages were cultured for the indicated times (in hours) in the absence (top) or presence (bottom) of zymosan. Proteins in the high-speed supernatant fraction of cell lysates (‘Intracellular’, left) or secreted into culture media (‘Extracellular’, right) were concentrated and probed for OM and β-tubulin by western blotting. FIG. 5C is a collection of two photographs which indicate oncomodulin mRNA expression visualized by RT-PCR. MΦ−, MΦ+: macrophages without or with zymosan treatment. Retinas, optic nerves, lens and superior colliculus (‘Sup coll’) were examined at postnatal day 2 (P2) or in adults (‘Ad’) without (−) or with (+) inflammatory response following lens injury (for retinas) or nerve crush (for optic nerves). FIG. 5D is a photograph of a western blot, indicating detection of oncomodulin in the retina by western blots (‘C’, normal control retina; ‘LI’, retina one week after lens injury). FIG. 5E is a photograph of westernblots, which indicates that preadsorbing anti-oncomodulin IgGs from antiserum diminishes oncomodulin staining on western blots. FIG. 5 F is a collection of nine photographs of cells in situ. The photographs show oncomodulin immunostaining in situ. Sections through the retina of a normal control or 1 week after activating macrophages (by lens injury) stained with antibody ED1 (for activated monocytes, red) and with either anti-oncomodulin (green) or preadsorbed anti-oncomodulin (‘Pre-ads’, to verify specificity of staining). Merged image shows oncomodulin-specific immunostaining in the ganglion cell layer (gcl) and inner plexiform layer (ipl) of the retina 1 week after lens injury. Scale bar, 50 μm.

FIG. 6 Oncomodulin promotes optic nerve regeneration in vivo. FIGS. 6A and 6B are photographs of longitudinal sections through the optic nerve immunostained to detect GAP-43⁺ axons distal to the injury site (asterisk) 2 weeks after nerve crush. Rats injected intraocularly with PLGA microspheres alone (FIG. 6A) or with microspheres containing oncomodulin plus sp-8-Br-cAMPs (FIG. 6B). Scale bar, 250 μm. FIG. 6C is a bar graph of data that quantitates axon growth ≧500 μm (light bars) and >1 mm (dark bars) distal to the injury site. FIG. 6D is a bar graph of data which indicates the length of longest axons (mm distal to injury site, averaged across all cases). *P<0.05, **P<0.01 and ***P<0.001, increase relative to blank microsphere-injected controls. ^{†}P<0.001, difference from group treated with sp-8-Br-cAMPs.

FIG. 7 Oncomodulin stimulates neurite outgrowth in DRG neurons. FIG. 7A is a collection of six photographs of variously treated DRG neurons. FIG. 7A-C Oncomodulin (OM) or saline was injected into DRGs in vivo 1 week before culturing cells on a permissive (poly-D-lysine+laminin) or nonpermissive (CSPG) substrate. FIG. 7A shows DRG neurons in culture stained with Tuj1 antibody. Scale bar, 100 μm. FIG. 7B-7D are bar graphs. FIG. 7B shows quantitation of neurite outgrowth on a permissive substrate. ***P<0.001, increase relative to saline-treated controls. FIG. 7C shows quantitation of outgrowth on a nonpermissive substrate. Oncomodulin and chondroitinase ABC (ChABC) each promoted some outgrowth (**P<0.01 relative to negative control); combining the two had a synergistic effect (^{††}P<0.01, ^{†††}P<0.001). FIG. 7D shows naïve DRG neurons incubated in the presence or absence of oncomodulin and forskolin as indicated. *P<0.05, increase above negative control; ^{†}P<0.05, increase relative to cells treated without forskolin.

### DETAILED DESCRIPTION

The present invention provides methods and compositions for preventing and/or treating peripheral nerve damage (peripheral neuropathy) in a subject. The method comprises administering oncomodulin to the subject. Optionally, additional factors (axogenic factors, and/or cAMP modulators and/or kinase inhibitors) are also administered. The amount of the factor(s) to be administered is a therapeutically effective amount.

The method may further comprise selecting a subject in need of treatment or prevention of peripheral nerve damage. Such selection may involve identification within a subject of peripheral nerve damage and/or identification of a risk for the development of peripheral nerve damage in the subject.

The compositions described herein can be used specifically in the methods described herein to treat damage associated with peripheral neuropathies including, but not limited to, the following: diabetic neuropathies, virus-associated neuropathies, including acquired immunodeficiency syndrome (AIDS) related neuropathy, infectious mononucleosis with polyneuritis, viral hepatitis with polyneuritis; Guillian-Barre syndrome; botulism-related neuropathy; toxic polyneuropathies including lead and alcohol-related neuropathies; nutritional neuropathies including subacute combined degeneration; angiopathic neuropathies including neuropathies associated with systemic lupus erythematosis; sarcoid-associated neuropathy; carcinomatous neuropathy; compression neuropathy (e.g. carpal tunnel syndrome) and hereditary neuropathies, such as Charcot-Marie-Tooth disease, peripheral nerve damage associated with spinal cord injury can also be treated with the present method. The subject is treated in accordance with the present method for peripheral nerve damage as the result of peripheral neuropathies, including those listed above. Subjects at risk for developing such peripheral nerve damage are also so treated.

Peripheral nerves such as dorsal root ganglia, otherwise known as spinal ganglia, are known to extend down the spinal column. These nerves can be injured as a result of spinal injury. Such peripheral nerve damage associated with spinal cord injury can also be treated using the present methods.

The injury for treatment can be acute or chronic. The spinal cord injury may be a complete severing of the spinal cord, a partial severing of the spinal cord, or a crushing or compression injury of the spinal cord. The spinal cord injury may have occurred more than three months prior to the treatment, more than one month prior, more than three weeks prior to the treatment, or more than two weeks prior to the treatment, more than one week prior to the treatment or from between 1-6 days prior to the treatment.

Administration of oncomodulin alone or in combinations described herein is to be made under conditions effective to stimulate nerve regeneration at the site of the injury and/or under conditions effective to at least partially restore nerve function through the injured spinal cord. Restoration of nerve function can be evidenced by restoration of nerve impulse conduction, a detectable increase in conduction action potentials, observation of anatomical continuity, restoration of more than one spinal root level, an increase in behavior or sensitivity, or a combination thereof. Administration is by a method which results in contacting the administered factors with the site of injury to thereby promote nerve regeneration (complete or partial).

Oncomodulin can be isolated according to the methods set forth in the examples and WO 01/091783, the disclosure of which is incorporated herein by reference. Active fragments, peptides, and portions of the molecule may also be used. Preferably the oncomodulin is derived (e.g. recombinant) from the species in which it is to be administered, such as human oncomodulin administered to a human subject. An example of a human oncomodulin cDNA is Genebank Accession NM 006188.

The term “axogenic factor” includes any factor that has the ability to stimulate axonal regeneration from a neuron. Examples of axogenic factors include AF-1 (mannose) and AF-2 as described in, for example, Schwalb et al. (1996) Neuroscience 72(4):901-10; Schwalb et al., id.; and U.S. Pat. No. 5,898,066, the contents of which are incorporated herein by reference. Other examples of axogenic factors include purines, such as inosine, as described in, for example, PCT application No. PCT/US98/03001, U.S. Pat. No. 6,440,455 and Benowitz et al. (1999) Proc. Natl. Acad. Sci. 96(23):13486-90, the contents of which are incorporated herein by reference.

A preferred axogenic factor in mannose (e.g., D-mannose or L-mannose) or a mannose derivative, e.g., aminomannose, mannose-6-phosphate (Phosporic acid mano-(3,4,5,6-tetrahydroxy-tetrahydro-pyran-2-ylmethy) ester).

A therapeutically effective amount or dosage of an axogenic factor may range from about 0.001 to 30 mg/kg body weight, with other ranges of the invention including about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, and 5 to 6 mg/kg body weight. For inosine, a non-limiting range for a therapeutically effective in vivo concentration in tissue containing the injury is 5 μM to 5 mM. These ranges and dosages are also envisioned for oncomodulin, although other doses and ranges may also be useful.

The term “cAMP modulator” includes any compound which has the ability to modulate, either up (increase) or down (decrease) the amount, production, concentration, activity or stability of cAMP in a cell, or to modulate the pharmacological activity of cellular cAMP. cAMP modulators may act at the level of adenylate cyclase, upstream of adenylate cyclase, or downstream of adenylate cyclase, such as at the level of cAMP itself, in the signaling pathway that leads to the production of cAMP. Cyclic AMP modulators may act inside the cell, for example at the level of a G-protein such as Gi, Go, Gq, Gs and Gt, or outside the cell, such as at the level of an extra-cellular receptor such as a G-protein coupled receptor. Cyclic AMP modulators include activators of adenylate cyclase such as forskolin; non-hydrolyzable analogues of cAMP including 8-bromo-cAMP, 8-chloro-cAMP, or dibutyryl cAMP (db-cAMP); isoprotenol; vasoactive intestinal peptide; calcium ionophores; membrane depolarization; macrophage-derived factors that stimulate cAMP; agents that stimulate macrophage activation such as zymosan or IFN-γ; phosphodiesterase inhibitors such as pentoxifylline and theophylline; specific phosphodiesterase IV (PDE IV) inhibitors; and beta 2-adrenoreceptor agonists such as salbutamol. The term cAMP modulator also includes compounds which inhibit cAMP production, function, activity or stability, such as phosphodiesterases, such as cyclic nucleotide phosphodiesterase 3B. cAMP modulators which inhibit cAMP production, function, activity or stability are known in the art and are described in, for example, Nano et al. (2000) Pflugers Arch 439(5):547-54, the contents of which are incorporated herein by reference.

“Phosphodiesterase IV inhibitor” refers to an agent that inhibits the activity of the enzyme phosphodiesterase IV. Examples of phosphodiesterase IV inhibitors are known in the art and include 4-arylpyrrolidinones, such as rolipram, nitraquazone, denbufylline, tibenelast, CP-80633 and quinazolinediones such as CP-77059.

“Beta-2 adrenoreceptor agonist” refers to an agent that stimulates the beta-2 adrenergic receptor. Examples of beta-2 adrenoreceptor agonists are known in the art and include salmeterol, fenoterol and isoproterenol.

The term “administering” to a subject includes dispensing, delivering or applying an active compound in a pharmaceutical formulation to a subject by any suitable route for delivery of the active compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route. Another form of administration suitable for treatment of spinal cord injury is injection into the spinal column or spinal canal.

As used herein, the language “contacting” is intended to include both in vivo or in vitro methods of bringing a compound of the invention into proximity with a neuron such that the compound can exert a neurosalutary effect on the neuron. In one embodiment, one or more of the factors described herein directly contact a neuron in need of regeneration. In another embodiment, one or more of the factors do not directly contact the neuron, but contact the surrounding cells. Combinations of different forms of contacting with the various factors described herein are also envisioned.

As used herein, a “neurosalutary effect” means a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The phrase “producing a neurosalutary effect” includes producing or effecting such a response or improvement in function or resilience within a component of the nervous system. For example, examples of producing a neurosalutary effect would include stimulating axonal outgrowth after injury to a neuron; rendering a neuron resistant to apoptosis; rendering a neuron resistant to a toxic compound such as .beta.-amyloid, ammonia, or other neurotoxins; reversing age-related neuronal atrophy or loss of function; or reversing age-related loss of cholinergic innervation.

As used herein, the term “effective amount” includes an amount effective, at dosages and for periods of time necessary, to achieve the desired result, such as sufficient to produce a neurosalutary effect in a subject. An effective amount of an active compound as defined herein may vary according to factors such as the disease state, age, and weight of the subject, and the ability of the active compound to elicit a desired response in the subject. Dosage regimens may be adjusted to provide the optimum therapeutic response. An effective amount is also one in which any toxic or detrimental effects of the active compound are outweighed by the therapeutically beneficial effects.

The term “subject” is intended to include animals. In particular embodiments, the subject is a mammal, a human or nonhuman primate, a dog, a cat, a horse, a cow or a rodent.

The route of administration and the dosage regimen will be determined by skilled clinicians, based on factors such as the exact nature of the condition being treated, the severity of the condition, and the age and general physical condition of the patient. The composition may be administered systemically, locally injected or delivered by topical or oral means. In one embodiment, the active compound formulation is administered into a subject intrathecally.

The oncomodulin may be contained in various types of pharmaceutical compositions, in accordance with formulation techniques known to those skilled in the art. For example, the compounds may be included in tablets, capsules, solutions, suspensions, and other dosage forms adapted for oral administration; and solutions and suspensions adapted for parenteral use. An appropriate buffer system (e.g., sodium phosphate, sodium acetate or sodium borate) may be added to prevent pH drift under storage conditions.

For injection, the active compound formulation of the invention can be formulated in liquid solutions, preferably in physiologically compatible buffers such as Hank's solution or Ringer's solution. In addition, the active compound formulation may be formulated in solid form and re-dissolved or suspended immediately prior to use. Lyophilized forms are also included. The injection can be, for example, in the form of a bolus injection or continuous infusion (such as using infusion pumps) of the active compound formulation.

In general, the doses used for the above described purposes will vary, but will be in an effective amount to prevent, reduce or ameliorate nerve damage resulting from any of the above listed conditions. As used herein, the term “pharmaceutically effective amount” refers to an amount of oncomodulin such that treatment of a patient with that amount can be associated with a medically desirable change in nerve function, or that can prevent, reduce, or ameliorate peripheral damage.

Experiments detailed in the Examples section below indicate that inhibition of PI3kinase, MEK, and JAK, potentiates the axogenic effects of oncomodulin. This indicates that administration of one or more such inhibitors (e.g. PD98059 for MEK inhibition, LY294002 for PI3K, and Jaki for JAK) with oncomodulin in the methods of the present invention will be useful and provide therapeutic effects to the subject.

It may further be useful to inhibit the effects of oncomodulin (e.g. the axogenic and/or neurosalutary effects) in a subject or in vitro or ex vivo. One such use is to regulate a therapeutic treatment for nerve damage in the subject, to contain the nerve growth response to the desired area. As such, another aspect of the present invention relates to a method for inhibiting oncomodulin in a subject in need thereof by administering an inhibitor of oncomodulin to a subject. One such inhibitor of oncomodulin is an inhibitor of CaMKII (e.g. KN92). Administration would be to a subject to promote contact to a region where neuronal outgrowth (the axogenic effect) promoting effect of oncomodulin is undesired. The method may first comprise identifying a subject in need of inhibition of oncomodulin. Such identification may involve determining the undesired presence of oncomodulin in a subject (or a region of a subject) wherein the effects of oncomodulin are not desired. A region of undesired presence of oncomodulin in the individual may be a region directly adjacent to nerve damage where oncomodulin therapy (e.g. administration) is planned or ongoing. Another such region may be an area of naturally or unnaturally occurring overexpression, or otherwise caused overactivity, of oncomodulin in a subject where the oncomodulin is causing undesired effects in the individual.

Another aspect of the present invention relates to a method for inhibiting oncomodulin in vitro or ex vivo by administering/contacting an inhibitor of oncomodulin to a neuron in vitro or ex vivo.

There is also provided an article of manufacture comprising packaging material and a pharmaceutical agent contained within the packaging material. The packaging material comprises a label which indicates that the pharmaceutical may be administered, for a sufficient term at an effective dose, for treating and/or preventing damage to peripheral nerves including damage resulting from ischemic or hypoxic stress, excess pressure, or injury. The pharmaceutical agent comprises neurotrophic compounds (oncomodulin optionally with axogenic factors and/or cAMP modulators) of the present invention together with a pharmaceutically acceptable carrier.

Also provided is an article of manufacture comprising packaging material and a pharmaceutical agent contained within the packaging material. The packaging material comprises a label which indicates that the pharmaceutical may be administered, for a sufficient term at an effective dose, for treating and/or preventing damage to the spinal cord or nerve damage resulting from stroke. The pharmaceutical agent comprises neurotrophic compounds (oncomodulin optionally with axogenic factors and/or cAMP modulators) of the present invention together with a pharmaceutically acceptable carrier.

As used herein, the term “pharmaceutically acceptable carrier” refers to any formulation which is safe, and provides the appropriate delivery for the desired route of administration of an effective amount of at least one compound of the present invention.

Oncomodulin may be localized at the site of the nerve damage by any suitable means. For example, it can be localised at the damage site within a matrix, e.g. a gel or solid.

Preferably, oncomodulin is localized at the damage site by means of a conduit around the nerve at the damage site. This is especially preferred where it is desired to bridge a gap in a severed nerve. However, other approaches may be better where the nerve is not severed, but rather damaged or degenerating. One example of such a condition is neuropraxia.

A conduit may be placed around the nerve damage site. The presence of the conduit per se may encourage nerve damage repair but the localisation of oncomodulin by the conduit will enhance this.

The conduit may be composed of any suitable material. For example, it may be composed of a non-bioabsorbable material such as silicone, which has been widely used in the past.

However, bioabsorbable materials are preferred, as they can be absorbed by the body when the damage is repaired. Collagen conduits (available from Integra Life Sciences) are one option in this respect.

In one respect, the present invention relates to the herein described compositions, methods, and respective component(s) thereof, as essential to the invention, yet open to the inclusion of unspecified elements, essential or not. In some embodiments, other elements to be included in the description of the composition, method or respective component thereof are limited to those that do not materially affect the basic and novel characteristic(s)” of the invention. This applies equally to steps within a described method as well as compositions and components therein. In other embodiments, the inventions, compositions, methods, and respective components thereof, described herein are intended to be exclusive of any element not deemed an essential element to the component, composition or method.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages may mean 1%.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.

### EXAMPLES

### Results

### Identification of Oncomodulin

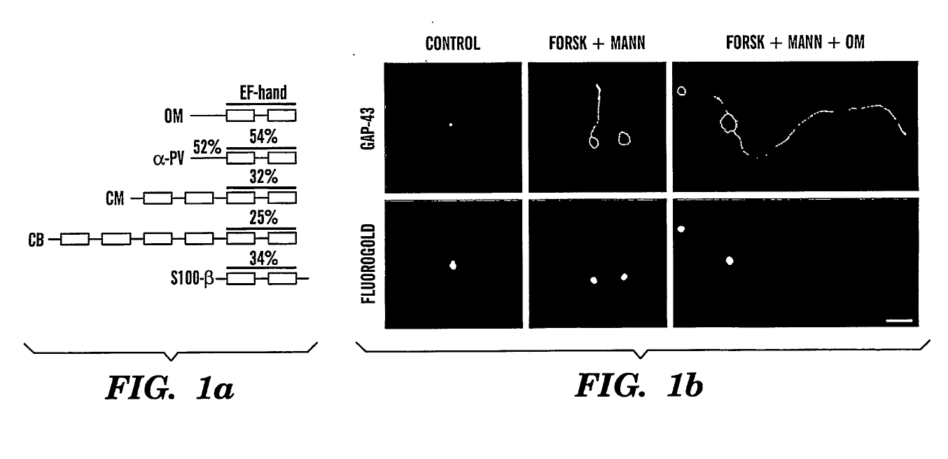

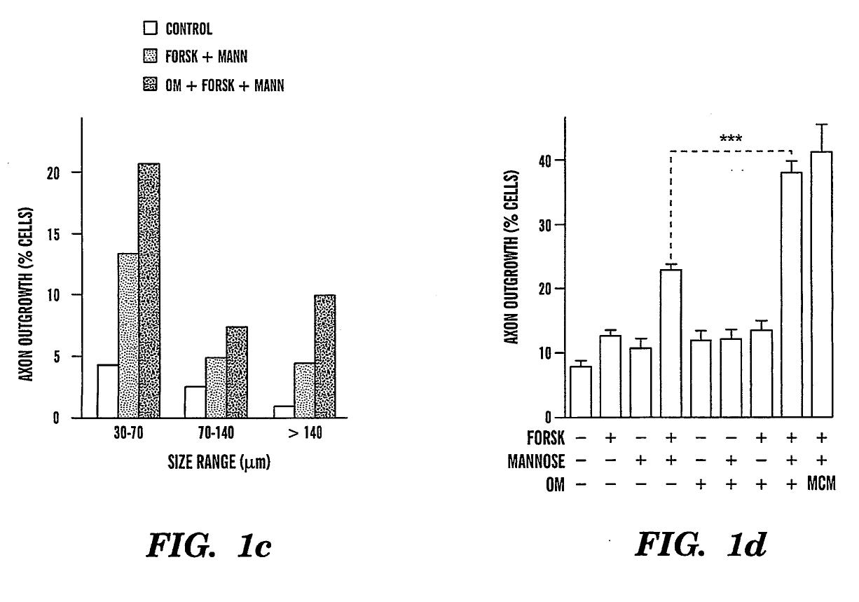

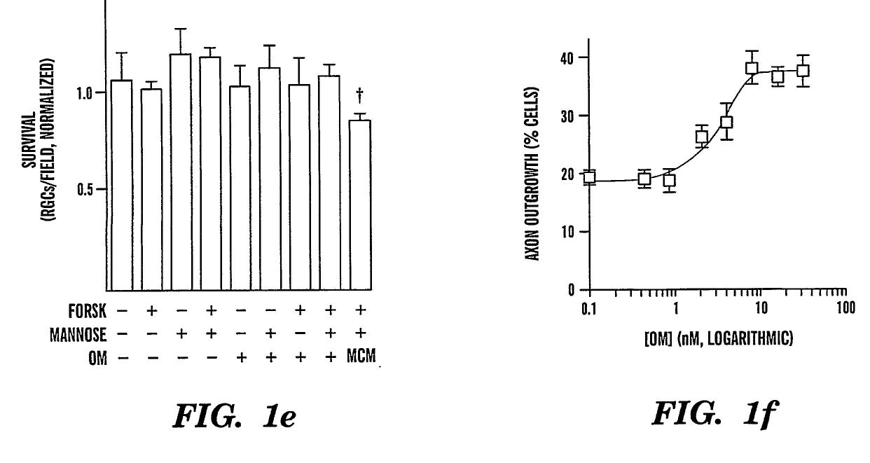

We previously found that when proteins secreted by activated macrophages are separated by size-exclusion chromatography, fractions containing proteins less than 20 kDa promote axon regeneration. These fractions contain a prominent band of 10-15 kDa (ref. 21), which was analyzed in the present study by mass spectrometry (Harvard Taplin Mass Spectrometry Facility). This analysis revealed the presence of the peptide (K)SLM*DAADNDGDGK (SEQ ID NO: 1), which is found in the protein oncomodulin. Oncomodulin is an 11.7-kDa Ca²⁺-binding protein that was named on the basis of its expression in several tumors and its limited resemblance to calmodulin²⁴. Oncomodulin has been highly conserved across vertebrate evolution (NCBI database) and includes a 40-residue N-terminal domain with a vestigial, inactive Ca²⁺-binding site (residues 7-33) and a 70-residue consensual EF-hand domain (FIG. 1a). This latter domain contains one site with relatively low Ca²⁺ and Mg²⁺ affinity (residues 41-70)₂₅ and a high-affinity Ca²⁺-binding site (residues 81-108). The only reported connection of oncomodulin to the nervous system has been its presence in hair cells of the inner ear²⁶. Mass spectrometry showed that lysozyme was also present in the 10-15 kDa band.

### Oncomodulin is a Potent Axon-Promoting Factor for RGCs

In keeping with earlier observations²², the combination of mannose and forskolin stimulated RGCs to extend axons in culture (FIG. 1b,c; P<0.0001). After 3 d, most axons were 30-70 μm in length, though some were 70-140 μm and a few were more than 140 μm. The addition of oncomodulin (OM) nearly doubled the amount of outgrowth (P<0.0001), particularly in the longest size range. Because forskolin, mannose and oncomodulin cause the entire axon growth histogram to shift to the right (FIG. 1c), we routinely collapsed the data across the three size classes to get a single, robust measure of outgrowth that allowed us to represent the effects of multiple experimental groups at once (for example, FIG. 1d). In the presence of forskolin and mannose, the effects of oncomodulin and MCM (prepared from zymosan-stimulated macrophages) were equivalent (FIG. 1d). None of these agents increased cell survival, though MCM was slightly toxic to RGCs (FIG. 1e). The effective concentration for half-maximum response (EC₅₀) of oncomodulin is ˜3.8 nM (FIG. 1f).

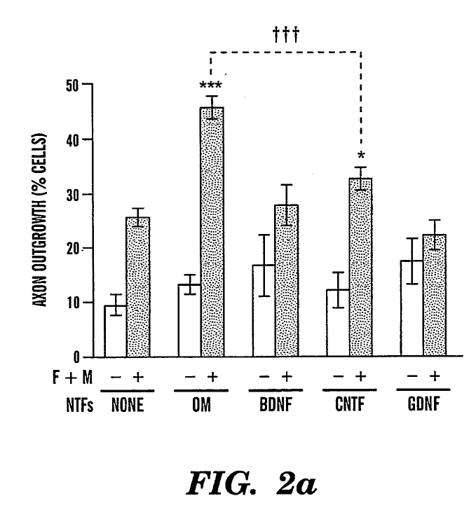

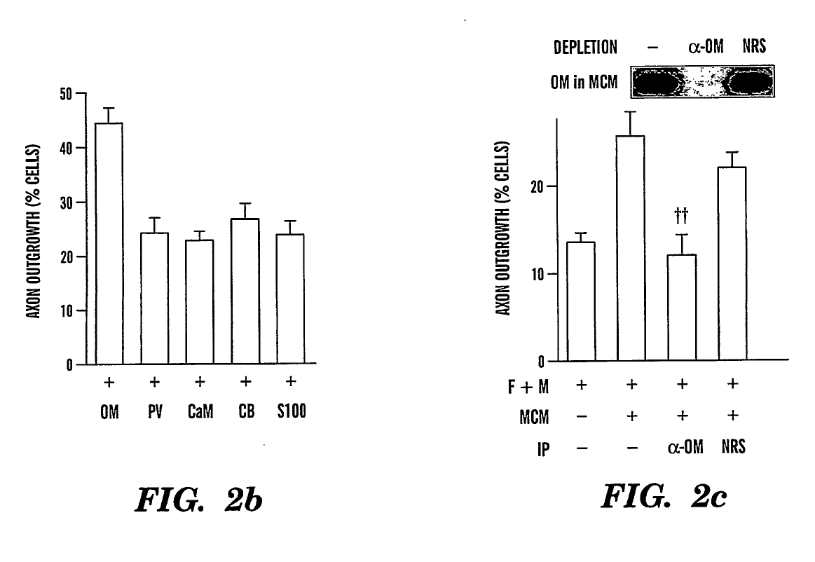

We compared the effect of oncomodulin to that of other growth factors known to enhance outgrowth and/or survival in RGCs—that is, ciliary neurotrophic factor (CNTF), brain-derived neurotrophic factor (BDNF) and glia-derived neurotrophic factor (GDNF)^{7-9,27-30}. Neither oncomodulin nor any of the other factors stimulated outgrowth in the absence of forskolin and mannose (FIG. 2a). In the presence of forskolin and mannose, CNTF increased axon growth (P<0.05), as expected^{28,30}, but to a significantly lesser extent than oncomodulin (FIG. 2a, P<0.001). BDNF and GDNF were ineffective.

### Specificity of Oncomodulin Effects

Ca²⁺-binding proteins with significant homology to oncomodulin include α-parvalbumin, calmodulin, calbindin and S100-β. These proteins were all inactive in our bioassay when tested at the same concentration as oncomodulin (15 nM, FIG. 2b) or at a tenfold higher concentration; S100-β showed some activity at 100 times this concentration (data not shown). Neither oncomodulin nor any other factor enhanced RGC survival above baseline, possibly due to the presence of survival factors in our culture media, such as insulin²⁷. Lysozyme, which copurified with oncomodulin, diminished RGC survival by ˜25% and did not enhance outgrowth (data not shown).

Adsorption of Oncomodulin from MCM Eliminates Activity

Unlike oncomodulin, proteins secreted by zymosan-stimulated macrophages exert some effect on RGCs even in the absence of forskolin and mannose²¹. This observation raises the question of whether oncomodulin is the principal axon-promoting factor secreted by zymosan-stimulated macrophages or whether additional growth factors are present. To investigate this issue, we first adsorbed the IgG fraction from a polyclonal rabbit anti-oncomodulin antiserum or from normal rabbit serum onto protein A beads, then used these beads to remove oncomodulin from MCM. Adsorbing MCM with the specific IgG reduced oncomodulin levels and eliminated axon-promoting activity (FIG. 2c); adsorption of MCM with normal rabbit IgG had only a minor effect. These findings show that oncomodulin is necessary for the biological activity of MCM and that, although additional growth-promoting factors may be present, their activity cannot be detected in the absence of oncomodulin.

### Oncomodulin Exhibits High-Affinity Binding to RGCs

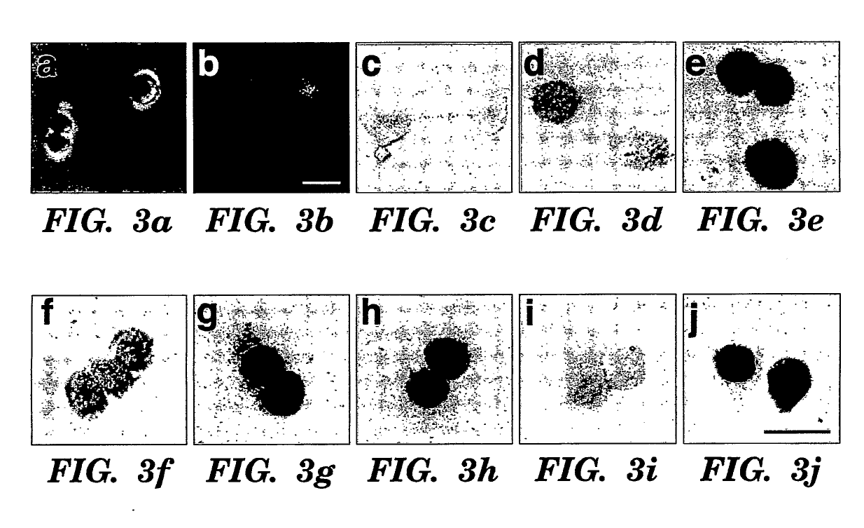

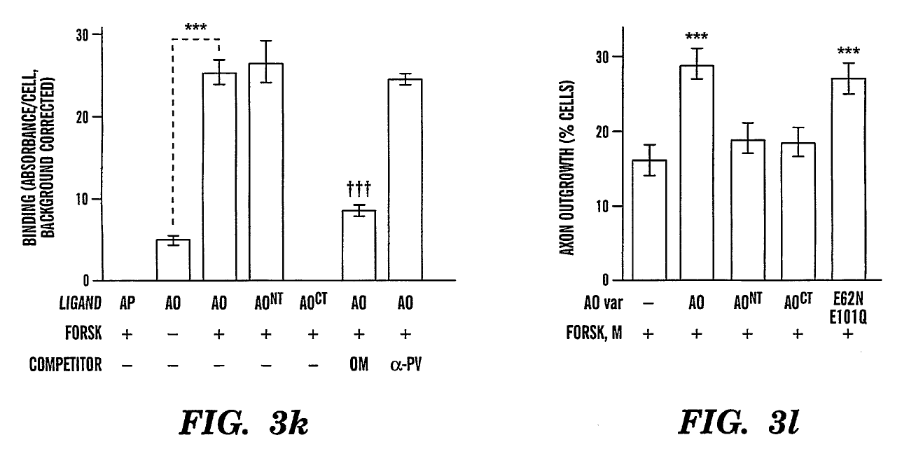

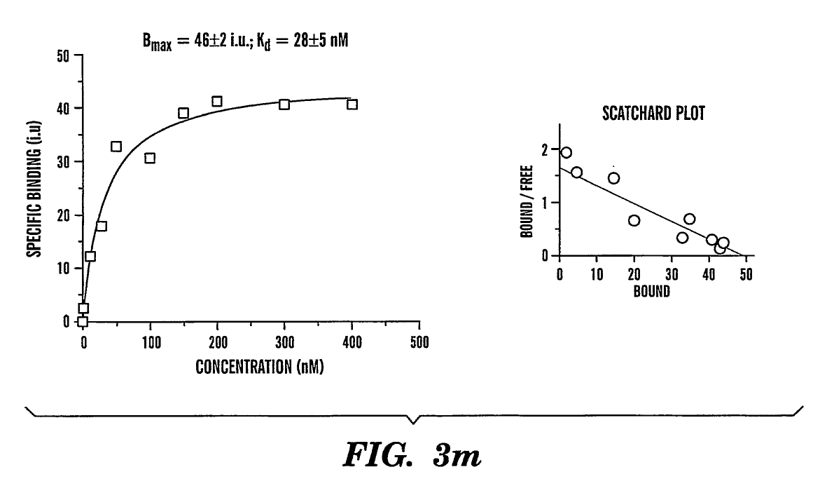

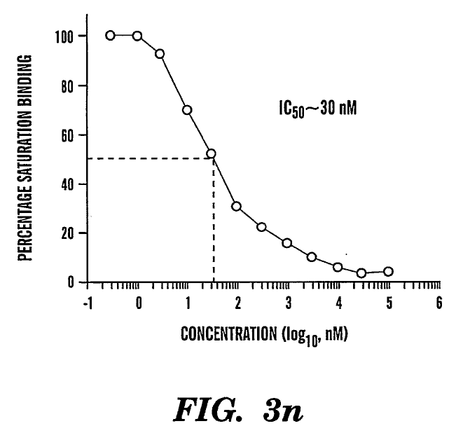

The finding that RGCs respond to low nanomolar concentrations of oncomodulin but not to related proteins suggests that oncomodulin may exert its effects through a high-affinity receptor. To investigate this possibility, we carried out receptor-ligand binding assays using RGCs that were purified by immunopanning³¹. Purity was estimated to be ˜98%, as evaluated using retrograde transport of Fluorogold to prelabel RGCs (FIG. 3a,b). After 1416 h in culture (in the presence or absence of forskolin), RGCs were lightly fixed and incubated with either an alkaline phosphatase (AP)-oncomodulin filsion protein (AP-OM) or recombinant AP alone. Neither AP-OM nor AP showed appreciable binding under basal conditions. However, AP-OM, but not AP, bound strongly to RGCs when the intracellular cAMP concentration ([cAMP]ᵢ) was elevated with forskolin (FIG. 3c-e) or with 8-bromoadenosine 3′,5′-cyclic monophosphate (sp-8-Br-cAMPs; data not shown). cAMP-dependent binding became evident at low nanomolar concentrations of oncomodulin and was strong at 10 nM (FIG. 3c-k), a concentration that results in strong outgrowth (FIG. 1f).

Whereas the C terminus of oncomodulin contains two active Ca²⁺-binding motifs, the N terminus contains evolutionarily conserved sequences not found in other proteins in the NCBI protein database. To investigate which domains are required for binding and bioactivity, we designed plasmids encoding alkaline phosphatase linked to either the N-terminal 50 amino acids of oncomodulin (AO^{NT}) or the C terminus (AO^{CT}); we also designed plasmids encoding AP linked to oncomodulin variants with single amino acid substitutions that substantially diminish Ca²⁺ affinity in the first binding site (AO^{E62N}), the second binding site (AO^{E101Q}) or both (AO^{E62N,E101Q}) (refs. 33,34). Whereas the binding of oncomodulin to RGCs required only the N terminus (FIG. 3h,i,k), biological activity required the presence of both the N and C termini (FIG. 3l). We were surprised to find that bioactivity was only slightly diminished by mutating E62 and E101 (FIG. 3l). Thus, the N terminus of oncomodulin is required for it to bind to RGCs, whereas the C terminus is also required for its biological activity.

Oncomodulin binding saturated at concentrations greater than 100 nM, with a dissociation constant (K_{d}) of 28±5 nM (FIG. 3m). Excess unconjugated oncomodulin displaced AP-OM from RGCs (FIG. 3f,k,n), but a 100-fold excess of parvalbumin did not (FIG. 3g,k). The half-maximal inhibitory concentration (IC₅₀) calculated from our displacement study (˜30 rinM, FIG. 3n) was nearly identical to the K_{d}. Thus, oncomodulin shows specific, high-affinity binding to RGCs that is saturable, reversible and cAMP dependent. When untreated RGCs were permeabilized after light fixation, AP-OM bound even without previous forskolin treatment (FIG. 3j). This observation suggests that cAMP causes the translocation of a receptor from the cytosol to the cell surface³².

### Mechanism of Oncomodulin Action

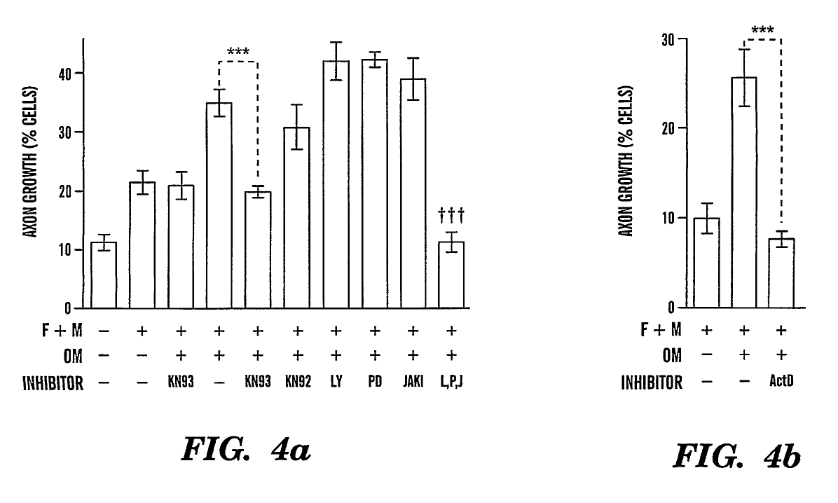

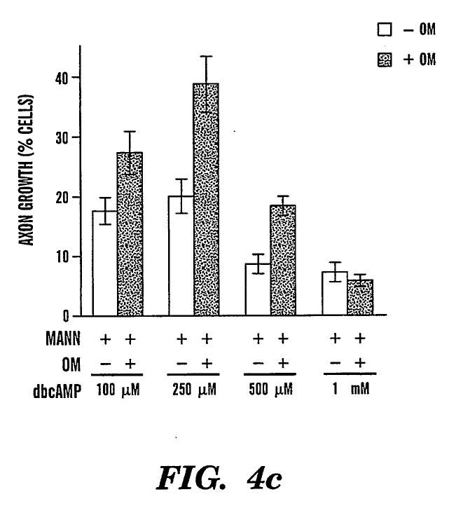

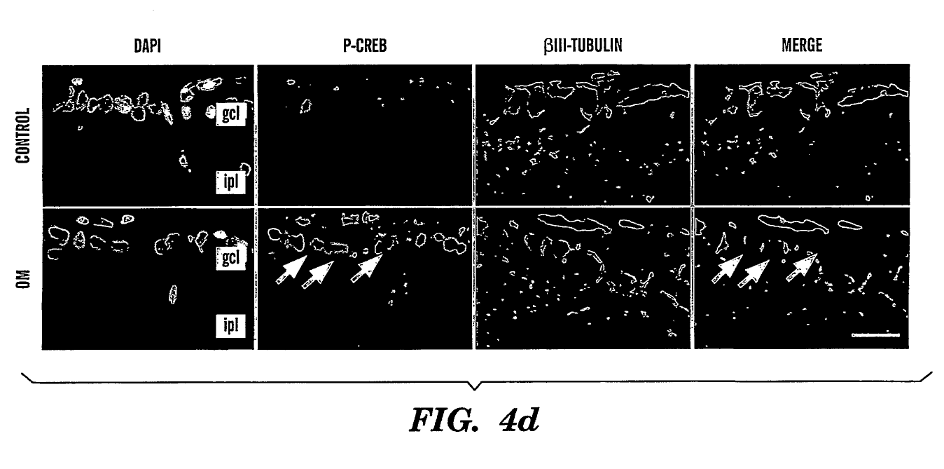

Agents that block signaling through Trk receptors or gp130 do not inhibit the pro-regenerative effects of intravitreal macrophage activation²³. Thus, if oncomodulin is essential for the positive effects of macrophage activation, we would predict that it acts via a signal transduction pathway distinct from those activated by neurotrophins or CNTF family members. In conformity with this prediction, the effects of oncomodulin were not blocked by inhibitors of MAP kinase kinase (MEK)-1, MEK-2 and MEK-5 (PD98059 at 5 μM) or of PI3 kinase (LY 294002 at 20 μM), which are activated by neurotrophins via Trk receptors; PD98059 enhanced outgrowth slightly (P<0.02). Blocking janus kinases (using Jakᵢ at 20 nM), which are activated downstream of CNTF family members, likewise did not diminish oncomodulin's activity (FIG. 4a). The combination of all three inhibitors strongly decreased outgrowth (FIG. 4a). This finding is not informative regarding oncomodulin signaling, however, as the three inhibitors also blocked the effect of forskolin plus mannose (data not shown), a prerequisite for oncomodulin's activity. In contrast to the other agents tested, KN93 (10 μM), an inhibitor of Ca²⁺/calmodulin-dependent protein kinase II (CaMKII), blocked the effect of oncomodulin fully without altering the effect of mannose and forskolin. KN92 (10 μM), an inactive analog of KN93, did not substantially diminish outgrowth (FIG. 4a). None of the agents used altered RGC survival (data not shown). In sum, oncomodulin signaling requires CaMKII but not MEK-1, MEK-2 or MEK-5, P13 kinase, or Jak-1, Jak-2 or Jak-3.

Intravitreal macrophage activation alters the expression of genes associated with axon outgrowth in RGCs (ref. 15). In keeping with this observation, actinomycin D (ActD, 8 nM), a transcriptional inhibitor, blocked the effects of oncomodulin on outgrowth (FIG. 4b) without altering RGC survival (data not shown).

The effects of trophic factors in overcoming the inhibitory effects of myelin are mediated through elevation of [cAMP]ᵢ; (ref. 35). The effects of oncomodulin on RGCs require elevation of [cAMP]ᵢ (FIG. 1d), and it is conceivable that once a minimal level of [cAMP]ᵢ is achieved, oncomodulin acts by elevating [cAMP]ᵢ further. In this case, we would expect that [cAMP]ᵢ elevation by itself should be sufficient to mimic the effects of oncomodulin and, conversely, that the effects of oncomodulin should not exceed those of high [cAMP]ᵢ. However, we found the opposite to be true. Whereas increasing the concentration of dibutyryl cAMP ([dB-cAMP]) up to 250 μM enhanced the effect of oncomodulin, further increases diminished outgrowth (FIG. 4c). Thus, although cAMP is required for the effects of oncomodulin, oncomodulin does not act by increasing [cAMP]ᵢ further.

The effect of macrophage activation on RGC gene expression, together with the inhibitory effects of ActD, suggest that oncomodulin activates a transcriptional cascade. Accordingly, injecting oncomodulin directly into the vitreous markedly increased the level of phosphorylated cAMP/Ca²⁺-response element binding protein (P-CREB), the active form of the transcriptional activator, in RGCs (FIG. 4d). Although the details of the downstream effects of oncomodulin are beyond the scope of the present study, our results indicate that it acts via a CaMKII-dependent pathway and involves transcriptional changes.

### Oncomodulin Secretion in Culture and In Vivo

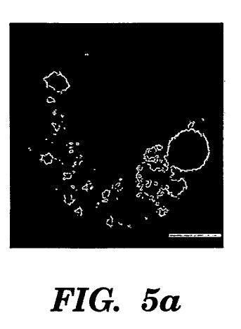

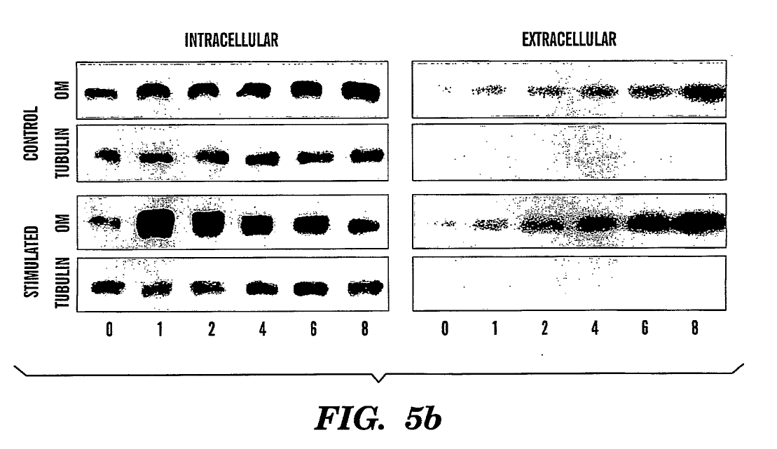

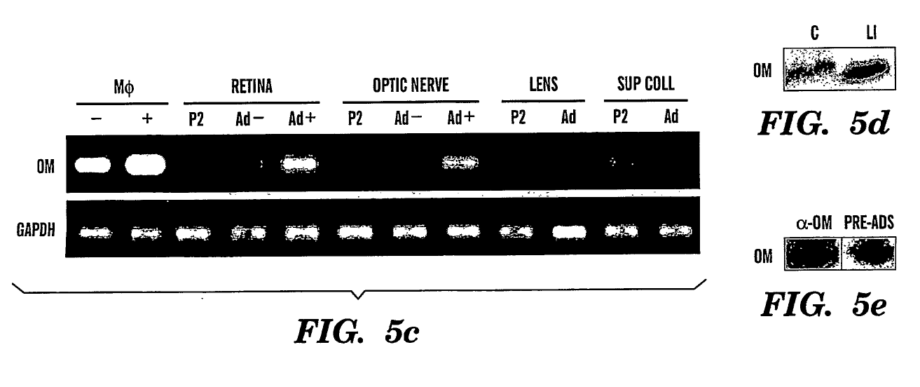

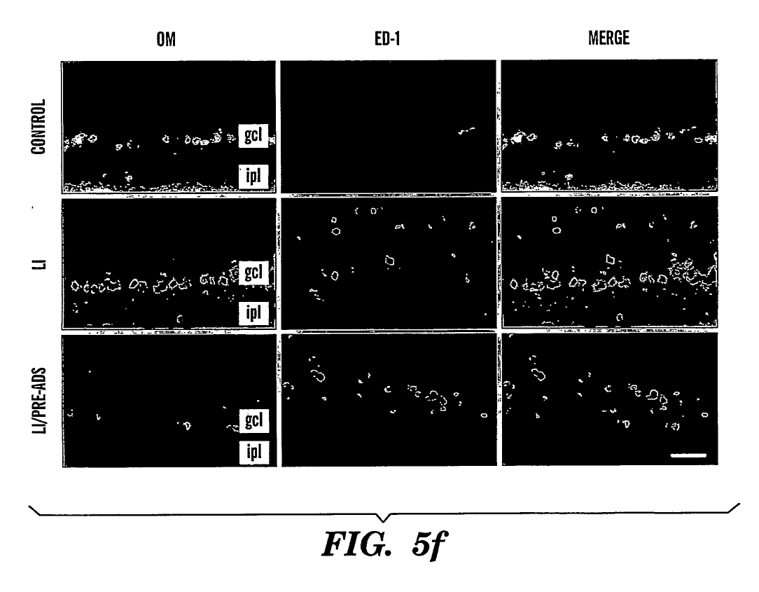

Oncomodulin does not include a consensual signal peptide sequence, raising the question of whether it is truly secreted from macrophages or whether its appearance in culture medium results from cell lysis. As demonstrated by confocal microscopy, the protein is concentrated in vesicles within macrophages (FIG. 5a), and in culture, it is secreted continuously (FIG. 5b). Zymosan, an activator of macrophages, increased the intracellular concentration of oncomodulin and its secretion (FIG. 5b). In contrast, β-tubulin, one of the most abundant cytosolic proteins did not increase in response to zymosan, nor did it appear extracellularly over the 8-h incubation period (FIG. 5b). Superoxide dismutase (SOD), another cytosolic protein expressed in macrophages³⁶, was also not detected extracellularly. Thus, the extracellular appearance of oncomodulin seems to reflect a physiological secretion process.

We conducted further experiments to investigate the expression of oncomodulin in vivo. By reverse transcription-polymerase chain reaction (RT-PCR), we determined that oncomodulin mRNA was present at low levels in the adult retina and increased greatly during inflammation (resulting from lens injury, ‘LI’ in FIG. 5c). A similar increase was detected at the protein level (FIG. 5d). To investigate the anatomical localization of oncomodulin in vivo, we double-immunostained the retina 7 d after lens injury to detect activated macrophages (antibody ED1) and oncomodulin. As a control for the specificity of the staining, we preadsorbed the anti-oncomodulin antiserum with recombinant oncomodulin (FIG. 5e). Positive immunostaining was found to increase in the ganglion cell and inner plexiform layers of the retina after lens injury (FIG. 5f). This staining was diminished, but not eliminated, by preadsorption of the primary antibody (FIG. 5f).

Additional RT-PCR revealed that oncomodulin mRNA is present in the developing and mature optic nerve and increases after injury, paralleling the inflammatory response that occurs in the damaged nerve¹⁷. In the superior colliculus, a principal target of the optic nerve, oncomodulin mRNA is detected in young rats but not in adults. Thus, in addition to being secreted by activated macrophages in vivo, oncomodulin is expressed in a part of the visual system that might serve as source of the protein during RGC development.

### Oncomodulin Stimulates Optic Nerve Regeneration In Vivo

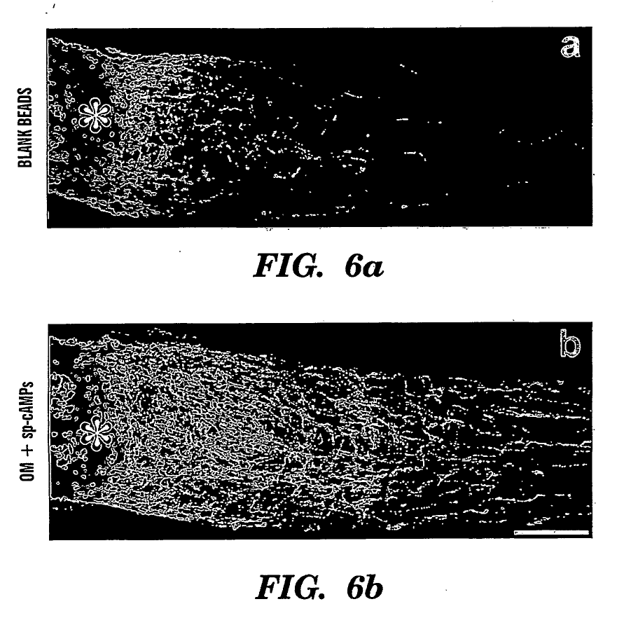

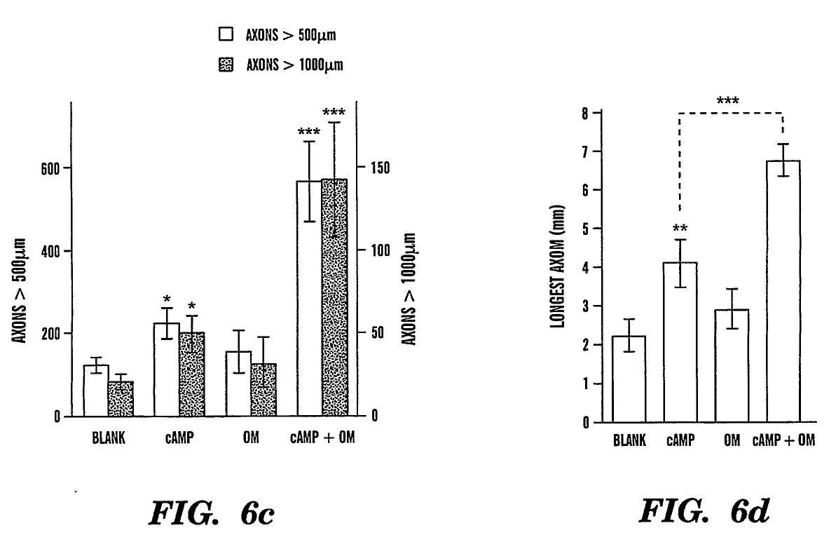

To investigate whether oncomodulin promotes axon regeneration in vivo, we delivered the protein, the cAMP analog 8-bromoadenosine 3′,5′-cyclic monophosphate (sp-8-Br-cAMP) or both into the vitreous after optic nerve injury using biocompatible, biodegradable poly-(lactic-co-glycolic acid) (PLGA) microspheres. After 2 weeks, controls injected with blank PLGA beads showed a small amount of regeneration past the injury site (FIG. 6a), which correlated with the influx of a small number of ED1⁺ macrophages into the eye (data not shown). Sp-8-Br-cAMPs alone increased outgrowth 2-fold (P<0.05) and oncomodulin by itself had no effect. In the presence of Sp-8-Br-cAMPs, however, oncomodulin increased regeneration into the distal optic nerve 5- to 7-fold over baseline (FIG. 6b,c; comparing the effects of oncomodulin+Sp-8-Br-cAMPs versus Sp-8-Br-cAMPs alone, P<0.001 for axons >500 μm in length, P<0.02 for axons >1 mm in length). Oncomodulin+Sp-8-Br-cAMPs did not increase the number of macrophages in the eye or alter the viability of RGCs compared to the cases with blank beads (P>0.5, data not shown).

We also investigated the effects of oncomodulin on the length of axon growth. Oncomodulin+Sp-8-Br-cAMPs significantly increased the length of the longest regenerating axons relative to Sp-8-Br-cAMPs alone (FIG. 6d, P<0.001).

### Oncomodulin Stimulates Outgrowth in DRG Neurons

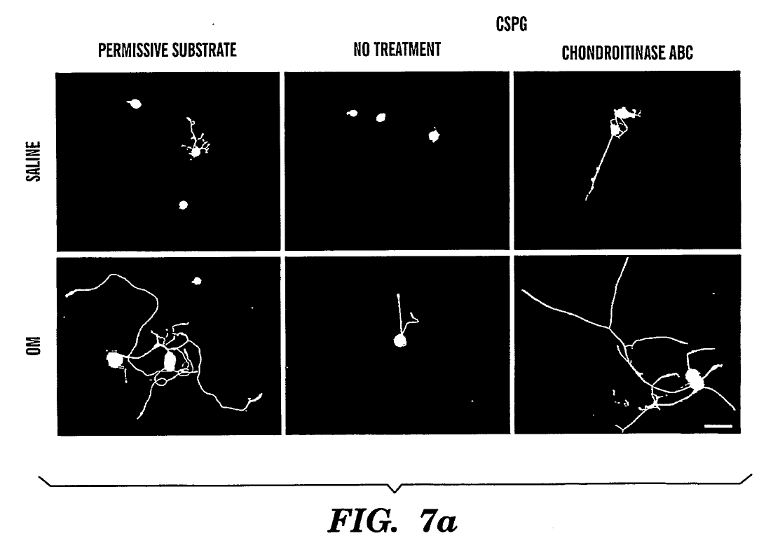

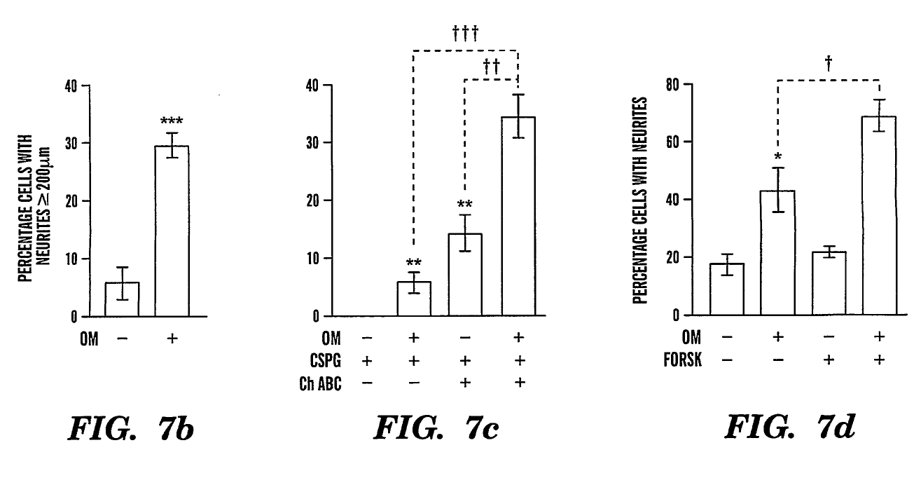

To investigate whether oncomodulin acts upon other cell populations, we used sensory neurons of the dorsal root ganglion (DRG). Intraganglionic macrophage activation greatly enhances the ability of DRG neurons to regenerate axons in vivo or when placed in culture 1 week later³⁷. We investigated whether intraganglionic oncomodulin injections could mimic some of these effects. DRG neurons exposed to oncomodulin in vivo for 1 week showed considerably greater outgrowth than vehicle-treated neurons when plated on a permissive poly-D-lysine substrate (FIG. 7a,b). On an inhibitory substrate containing chondroitin sulfate proteoglycans (CSPGs), DRG neurons pretreated with saline did not extend neurites. Oncomodulin pretreatment stimulated some outgrowth, and this effect was further enhanced by treating cultures with the enzyme chondroitinase ABC to degrade CSPGs (FIG. 7a,c).

Further experiments demonstrated that oncomodulin acts on previously untreated DRG neurons and that this effect is enhanced with forskolin (FIG. 7d), though not with mannose (data not shown). Although these experiments do not prove that oncomodulin accounts for the reported effect of macrophage activation on DRG axon regeneration³⁷, they do show that it can stimulate outgrowth in neuronal populations other than RGCs.

### DISCUSSION

Activated macrophages stimulate RGCs to regenerate axons through the injured optic nerve^{8,15,18,21,23}, enable DRG neurons to regenerate their central branches into the spinal cord^{37,38} and enhance functional recovery after spinal cord injury³⁹. The present study shows that oncomodulin, a 12-kDa Ca²⁺-binding protein, is a potent macrophage-derived growth factor for RGCs and other neurons. We show for the first time that oncomodulin is abundantly expressed and secreted by macrophages, that it binds to RGCs with high-affinity and that, in the presence of mannose plus elevated cAMP, it activates a CaM kinase II-dependent pathway that leads to greater axon outgrowth than other known polypeptide growth factors. Immunodepletion of oncomodulin eliminated the axon-promoting effects of macrophages, and in vivo, continuous release of oncomodulin plus a cAMP analog enabled RGCs to regenerate axons into the highly inhibitory environment of the adult optic nerve.

Oncomodulin seems to act through a high-affinity cell-surface receptor. This protein shows saturable binding to RGCs with a K_{d} of 28±5 nM. This value exceeds the EC₅₀ value (˜3.8 nM) and may reflect the existence of ‘spare receptors’ on RGCs, as is found for other ligand-receptor pairs in neurons^{40,41}. The binding of oncomodulin to RGCs is reversible (IC₅₀˜30 nM) and highly specific. The most closely related Ca²⁺-binding protein, parvalbumin, does not compete with oncomodulin for receptor occupancy, and neither parvalbumin nor any other Ca²⁺-binding protein stimulated outgrowth from RGCs at concentrations up to 150 nM. The receptor binding site of oncomodulin lies in its N terminus, which contains highly conserved sequences not found in other Ca²⁺-binding proteins. The axon-promoting effects of oncomodulin also require the C-terminal EF-hand domain but are relatively insensitive to the attenuation of its Ca²⁺ affinity. Regarding the cAMP dependence of oncomodulin binding to RGCs, possible explanations include a role for cAMP in regulating the expression of the receptor (or a coreceptor); in activating a dormant receptor or coreceptor; or in regulating the translocation of a receptor or coreceptor from a cytosolic pool to the cell membrane, as is known to occur for trkb (refs. 32,42). Consistent with this latter possibility, permeabilizing RGCs enabled oncomodulin to bind even without elevating [cAMP]ᵢ, presumably to an intracellular pool of receptors. Although mannose is not required for the binding of oncomodulin to its receptor, it is nevertheless required for RGCs to respond to the protein. The basis for this requirement is presently unknown. In intact cells, oncomodulin does not seem to get internalized before binding to its receptor, as it binds to RGCs that had been fixed after being exposed to forskolin while alive. Whether the oncomodulin-receptor complex becomes internalized after binding is unknown. In sum, our studies point to the existence of a high-affinity oncomodulin receptor on RGCs that shows specificity, saturability and cAMP dependence.

Our findings raise the question of why RGCs might express a receptor for a macrophage-derived signal. The vitreous is highly resistant to inflammation and RGCs only encounter macrophages under unusual circumstances⁴³. It is possible that an alternate ligand exists for the receptor. However, the only protein containing sequences homologous to the N terminus of oncomodulin is α-parvalbumin, which does not compete for receptor occupancy. Another possibility is that RGCs normally respond to oncomodulin from cells other than macrophages. Along these lines, we found that a principal target of retinal axons, the superior colliculus, expresses oncomodulin during development. Further experiments will be required to determine whether there is another physiological source of oncomodulin for RGCs and to determine whether this protein is involved in cell-cell signaling outside the nervous system.

Oncomodulin differs in its activity and downstream signaling mechanisms from other polypeptide growth factors known to act on RGCs. Among previously studied growth factors, CNTF was the most effective in stimulating RGCs to extend axons in culture, and this effect was [CAMP] dependent^{28,30}. BDNF stimulates local sprouting, rather than long distance growth, from mature RGCs, and both BDNF and CNTF enhance survival^{28,29,44}. In our study, CNTF, but not BDNF or GDNF, enhanced outgrowth from RGCs in culture in the presence of mannose and forskolin, though its effect was less than that of oncomodulin. The effects of other growth factors in vivo have not been tested under the conditions used here. A previous study found that CNTF is ineffective in stimulating RGCs to regenerate axons into the mature optic nerve, but it was not delivered continuously nor in the presence of agents to elevate cAMP (ref. 18). In another experimental model, however, CNTF plus elevated cAMP enhanced RGC axon regeneration through a peripheral nerve graft³⁰ to approximately the same extent as intraocular macrophage activation²¹. In addition to the factors tested here, we previously reported that fibroblast growth factor-2 (FGF2), nerve growth factor (NGF), cardiotrophin, interleukin-6, epidermal growth factor (EGF) and several chemokines do not stimulate mature RGCs to extend axons in culture²¹.

In parallel to the differences observed in the bioactivity of oncomodulin versus other factors, there are marked differences in downstream signaling pathways. The effects of oncomodulin on RGCs were unaffected by agents that block the activity of PI3 kinase or MEK-1, MEK-2 or MEK-5, which are activated by BDNF and related neurotrophins, or by an inhibitor of janus kinases, which are activated by CNTF and related cytokines. In agreement with these findings, the pro-regenerative effects of intravitreal macrophage activation are reported to be insensitive to agents that interfere with the receptors for neurotrophins or CNTF family members²³. An inhibitor of CaMKII, however, blocked the effects of oncomodulin completely and selectively. Further downstream, the effects of oncomodulin were also blocked by a transcriptional inhibitor. This finding is consistent with in vivo observations that oncomodulin leads to the phosphorylation of the transcriptional activator CREB in RGCs and that macrophage activation induces the expression of genes related to axon outgrowth in RGCs (ref. 15). In sum, although the precise mechanisms linking oncomodulin to axon outgrowth remain to be clarified, our results show that it activates a signaling pathway that involves CaMKII activity and downstream transcriptional changes.

To determine whether oncomodulin stimulates outgrowth in any other neural populations, we investigated its effects on DRG sensory neurons. Macrophage activation in DRGs enhances these neurons' ability to regenerate axons when explanted in culture and through the dorsal root entry zone in vivo^{37,38}. Following the procedure used in the former study³⁷, we found that exposing sensory neurons to oncomodulin in vivo stimulated their growth when placed in culture and potentiated the effect of chondroitinase ABC in enabling these cells to grow on an inhibitory CSPG substrate. Oncomodulin promoted outgrowth from DRG neurons even in the absence of agents that elevate [cAMP]ᵢ, though forskolin augmented its effects. Although these results do not prove that oncomodulin mediates the effects of macrophage activation on sensory neuron regeneration, they do show that it can act on neural populations other than RGCs.

Although oncomodulin accounts for many of the effects of intravitreal macrophage activation, it cannot account for them all. Oncomodulin was isolated here as the major axon-promoting protein secreted by activated macrophages, and consistent with this, immunodepletion of this protein eliminated the activity of MCM on RGCs. However, whereas the axon-promoting effect of oncomodulin in vivo requires the presence of agents to elevate [cAMP]ᵢ, intravitreal macrophage activation alone causes more RGCs to extend axons 1 nun beyond the injury site than oncomodulin plus a cAMP analog (OM/cAMP: compare FIG. 6c with FIG. 2 in ref. 21). Some of this difference may be due to the strong effect of macrophage activation on RGC survival. On the other hand, the average length of the longest regenerating axons was somewhat greater in response to OM/cAMP (6.7 mm) than intravitreal macrophage activation (5.5 mm). Eye injury in the absence of macrophage activation causes no regeneration^{18,21}. In culture, oncomodulin requires mannose together with agents to elevate [cAMP]ᵢ in order to stimulate outgrowth from RGCs. MCM has a modest effect by itself, but in the presence of forskolin and mannose its effects are similar to those of oncomodulin. Together, these results suggest that although oncomodulin is essential for the axon-promoting effects of macrophages on RGCs, other factors produced by macrophages help promote cell survival and may augment the effects of oncomodulin on outgrowth, perhaps by increasing [cAMP]ᵢ. Additional issues that complicate any comparison between the effects of OM/cAMP and those of macrophage-derived factors include the possibility that the latter may cause the release of secondary agents from other cells in the retina that contribute to outgrowth, the unknown release characteristics of OM/cAMP from microspheres, and the rates at which OMIcAMP degrade or diffuse out of the eye. Also, as shown here and elsewhere^{5,30}, if cAMP levels used were too high, this would be deleterious. It is likely that the amount of regeneration obtained here can be substantially improved upon by controlling the delivery of oncomodulin and agents to elevate [cAMP]ᵢ more precisely, enhancing RGC survival^{10,11,45}, and by counteracting inhibitory signals associated with myelin and the glial scar^{13,15}. Such a combinatorial approach may ultimately result in levels of optic nerve regeneration that are clinically meaningful.

### Methods

All in vivo work in this paper was performed at the Children's Hospital with the approval of the Institutional Animal Care and Use Committee.

### Identification of Oncomodulin.

Oncomodulin was identified by high-performance liquid chromatography (HPLC) tandem mass spectrometry (LC-MS/MS) performed at the Harvard Microchemistry and Proteomics Analysis Facility. A protein band, Mᵣ˜14 kDa, was excised from an SDS-polyacrylamide gel containing proteins secreted by zymosan-stimulated macrophages after separation by size-exclusion chromatography. This was the most conspicuous band present in the column fractions that were found to stimulate axon outgrowth from retinal ganglion cells in culture²¹. Sequence analysis on tryptic peptides of the 14-kDa band was performed with microcapillary reverse-phase HPLC directly coupled to the nano-electrospray ionization source of an ion-trap mass spectrometer (Finnigan LCQ DECA XP). Resulting MS/MS spectra were correlated with known sequences using SEQUEST and programs developed in-house. Results were then manually confirmed for fidelity.

### Macrophage Culture and Oncomodulin Detection.

Rat macrophages were cultured in the presence or absence of zymosan as described²¹. Macrophage-conditioned media (MCM) was collected after incubating the cells for 1-8 h. Cells were collected at the same time intervals, homogenized, and high-speed supernatant fractions were prepared. Oncomodulin was visualized by western blotting using a monoclonal antibody²⁶ (1:5,000). In some experiments, we used immunofluorescence to visualize the protein in cultured macrophages (monoclonal antibody, 1:2,000) or in retinal sections (rabbit polyclonal antibody to oncomodulin (anti-OM), 1:2,000, Swant). Retinal sections were double-stained with antibody ED1 (1:200, Serotec) to detect macrophages. In all cases, appropriate fluorescent secondary antibodies (Molecular Probes) were used at 1:500. Controls included adsorption of anti-oncomodulin IgGs from the antiserum onto an oncomodulin-coated or a control nitrocellulose filter. The presence or absence of anti-oncomodulin antibodies was verified by western blotting.

Immunodepletion of Oncomodulin from Macrophage-Conditioned Medium.

Protein A beads (Sigma) were incubated with either rabbit anti-OM antiserum (Swant) or normal rabbit serum (Invitrogen) for 16 h at 4° C. After extensive rinsing, beads with adsorbed IgGs were mixed with MCM (1 ml 10× concentrate, collected 8 h after zymosan treatment) for 24 h at 4° C. and then pelleted down by centrifugation. We tested 5 μl of the supernatants or untreated MCM for oncomodulin by western blotting. Bioactivity of immune-depleted and control MCM was tested in dissociated RGC cultures as described below at a 1:4 dilution, a concentration found to give a near-maximal response.

### Primary Retinal Cultures.

Adult rat retinal cultures were prepared as described²¹. Briefly, RGCs were retrogradely labeled by injecting Fluorogold into the superior colliculus. One week later, retinas were dissected, dissociated, and the cells plated in defined, serum-free medium. Axon growth (% RGCs extending axons ≧30 μm, ≧70 μm or ≧140 μm in length) was evaluated after 3 d in quadruplicate samples in a blinded fashion. All experiments were repeated at least three separate times. In some instances, cells were immunostained with GAP-43 antibody (1:500, Chemicon).

### Immunopurified RGC Cultures.

Adult rat retinal cells were dissociated as above and then isolated by immunopanning^{31,46} using antibody MAC1 followed by an antibody to Thy1 (Chemicon). Purity was ˜98% based upon counting Fluorogold-labeled versus total cells. RGCs, treated with or without forskolin, were lightly fixed after 14-16 h in culture (4% paraformaldehyde (PFA), 6 min) before binding assays.

### Production of Recombinant Oncomodulin Proteins.

Rat oncomodulin was expressed in Escherichia coli and purified through DEAE-Sepharose and Sephadex G-75 columns, yielding recombinant oncomodulin with purity >98% as judged by SDS-polyacrylamide gel electrophoresis (SDS-PAGE) and ultraviolet absorbance⁴⁷. An alkaline phosphatase-oncomodulin (AP-OM) plasmid was generated by fusing the oncomodulin gene into vector pAP5 (gift from Z. He, Children's Hospital, Harvard Medical School, Boston). E62Q, E101N and E62Q+E101N mutant oncomodulin plasmids were made by single amino acid exchange at sites known to be critical for strong Ca²⁺-binding^{33,34} (site-directed mutagenesis kit, Stratagene). OM^{NT} and OM^{CT} are truncated variants of oncomodulin representing the N-terminal 50 amino acids and the C-terminal region, respectively. All mutated genes were inserted into pAP5. AP or AP-fusion plasmids were transfected into 293T cells. Recombinant proteins were purified using Ni-NTA columns (Qiagen) and verified by western blotting with antibodies to AP and, where possible, to oncomodulin.

### RT-PCR.

Total RNA from various rat tissues was extracted using RNeasy (Qiagen). First-strand cDNA was synthesized according to the manufacturer's instructions (Invitrogen). PCR was carried out using the first-strand cDNA as the template with rat oncomodulin primers (5′→3′: ATGAGCATCACGGACATCCTG (SEQ ID NO: 2); 3′→5′: AGAGTGCACCATTTCCTG (SEQ ID NO: 3)). PCR fragments were sequenced to verify that they correspond to oncomodulin.

### Ligand Binding Assay.

Binding assays were carried out as described⁴⁸ with slight modifications. Briefly, lightly fixed, immunopurified RGCs were incubated with AP-OM or AP (37° C., 24 h).

After extensive rinsing, cells were fixed again, heated (65° C., 90 min) to destroy endogenous AP and incubated with nitro blue tetrazolium chloride/5-bromo-4-chloro-3-indoyl phosphate toluidine salt (BCIPINBT). In some instances, AP-OM or AP (control), with or without forskolin, was added to the cultures before the initial fixation. Absorbance was measured with Image J software (US National Institutes of Health) and corrected by subtracting the level of AP binding. Binding curves and Scatchard plots were generated using Prism software. For displacement studies, varying concentrations of unlabeled oncomodulin were added to cultures after the equilibrium binding of 40 mM AP-OM at 37° C. for 24 h.

### Polymeric Microspheres.

Microspheres were prepared from lyophilized oncomodulin or sp-8-Br-cAMPs (Sigma) with PLGA using the solvent evaporation method of single emulsion⁴⁹. To measure the rate of protein release in vitro, 10 mg of oncomodulin-containing beads were incubated in phosphate-buffered saline (PBS; 37° C.). Supernatants were collected every 3 d and analyzed on western blots. Following an initial burst, low levels of oncomodulin were found to be released continuously over a 1-month period.

### Optic Nerve Surgery and Intravitreous Injections.

Optic nerve surgery was carried out on male Fisher rats (200-250 g) as described¹⁸. Three days after nerve crush, rats received intraocular injections of blank microspheres or microspheres containing oncomodulin, sp-8-Br-cAMPs or both, in 10 μl saline after the same fluid volume was withdrawn from the eye (n=8-12 per group). Rats were killed 2 weeks later and their retinas, with optic nerves attached, were prepared as described^{18,21}. Axon growth was evaluated by GAP-43 immunostaining in the optic nerve at distances of 500 μm and 1 mm from the injury site^{18,21}. The length of the longest axon was measured in each case and averaged across all cases within each group.

To investigate the immediate effects of oncomodulin in vivo, rats received an intravitreous injection of the protein (1 μg μl⁻¹, 5 μl volume, n=6) and were killed 2 h later. Retinal sections were immunostained to detect the phosphorylated form of the transcriptional activator CREB (antibody to P-CREB, 1:100, Cell Signaling Technology). Fluorescent photomicrographs were taken 2 mm from the optic nerve head.

### DRG Injections and Cultures.

L4-L5 DRGs from adult male Sprague-Dawley rats (250-300 g) were injected with 5 μl of saline or oncomodulin (200 ng μl⁻¹). After 7 d, injected ganglia were dissected and single-cell suspensions were prepared as described⁵⁰. Cells were cultured on laminin (Sigma) with or without CSPGs (Chemicon) for 20 h or 40 h. In some cases, chondroitinase ABC (0.5 U ml⁻¹, Seikagaku) was added. Neurite outgrowth was visualized by Tuj1 antibody to class III β-tubulin (1:5,000, Babco) and quantified in quadruplicate samples by a blinded observer. In other studies, L4-L5 DRG neurons were cultured in the presence of absence of oncomodulin, mannose and/or forskolin in RPMI-1640 to limit spontaneous outgrowth. Cells were fixed and immunostained after 3 d and evaluated for outgrowth.

The references cited herein are incorporated by reference.

### REFERENCES

1. Ramon y Cajal, S. Degeneration and Regeneration of the Nervous System (Oxford Univ. Press, New York, 1991).
2. Aguayo, A. J. et al. Degenerative and regenerative responses of injured neurons in the central nervous system of adult mammals. Philos. Trans. R. Soc. Lond. B Biol. Sci. 331, 337-343 (1991).
3. Chierzi, S., Strettoi, E., Cenni, M. C. & Maffei, L. Optic nerve crush: axonal responses in wild-type and bcl-2 transgenic mice. J. Neurosci. 19, 8367-8376 (1999).
4. Shen, S., Wiemelt, A. P., McMorris, F. A. & Barres, B. A. Retinal ganglion cells lose trophic responsiveness after axotomy. Neuron 23, 285-295 (1999).
5. Monsul, N. T. et al. Intraocular injection of dibutyryl cyclic AMP promotes axon regeneration in rat optic nerve. Exp. Neurol. 186, 124133 (2004).
6. Kermer, P., Klocker, N., Labes, M. & Bahr, M. Inhibition of CPP32-like proteases rescues axotomized retinal ganglion cells from secondary cell death in vivo. J. Neurosci. 18, 46564662 (1998).
7. Mey, J. & Thanos, S. Intravitreal injections of neurotrophic factors support the survival of axotomized retinal ganglion cells in adult rats in vivo. Brain Res. 602, 304317 (1993).

8. Pernet, V. & Di Polo, A. Synergistic action of brain-derived neurotrophic factor and lens injury promotes retinal ganglion cell survival, but leads to optic nerve dystrophy in vivo. Brain (2006).
9. Koeberle, P. D. & Ball, A. K. Effects of GDNF on retinal ganglion cell survival following axotomy. Vision Res. 38, 1505-1515 (1998).
10. Cheng, L., Sapieha, P., Kittlerova, P., Hauswirth, W. W. & Di Polo, A. TrkB gene transfer protects retinal ganglion cells from axotomy-induced death in vivo. J. Neurosci. 22, 3977-3986 (2002).
11. Zhou, Y., Pernet, V., Hauswirth, W. W. & Di Polo, A. Activation of the extracellular signal-regulated kinase ½ pathway by AAV gene transfer protects retinal ganglion cells in glaucoma. Mol. Ther. 12, 402-412 (2005).
12. Weibel, D., Cadelli, D. & Schwab, M. E. Regeneration of lesioned rat optic nerve fibers is improved after neutralization of myelin-associated neurite growth inhibitors. Brain Res. 642, 259-266 (1994).
13. Fischer, D., He, Z. & Benowitz, L. I. Counteracting the Nogo receptor enhances optic nerve regeneration if retinal ganglion cells are in an active growth state. J. Neurosci. 24, 1646-1651 (2004).
14. Lehmann, M. et al. Inactivation of Rho signaling pathway promotes CNS axon regeneration. J. Neurosci. 19, 7537-7547 (1999).
15. Fischer, D., Petkova, V., Thanos, S. & Benowitz, L. I. Switching mature retinal ganglion cells to a robust growth state in vivo: gene expression and synergy with RhoA inactivation. J. Neurosci. 24, 87268740 (2004).
16. Koprivica, V. et al. EGFR activation mediates inhibition of axon regeneration by myelin and chondroitin sulfate proteoglycans. Science 310, 106-110 (2005).
17. Berry, M., Carlile, J. & Hunter, A. Peripheral nerve explants grafted into the vitreous body of the eye promote the regeneration of retinal ganglion cell axons severed in the optic nerve. J. Neurocytol. 25, 147-170 (1996).
18. Leon, S., Yin, Y., Nguyen, J., Irwin, N. & Benowitz, L. I. Lens injury stimulates axon regeneration in the mature rat optic nerve. J. Neurosci. 20, 4615-4626 (2000).
19. Fischer, D., Heiduschka, P. & Thanos, S. Lens-injury-stimulated axonal regeneration throughout the optic pathway of adult rats. Exp. Neurol. 172, 257-272 (2001).
20. Lorber, B., Berry, M. & Logan, A. Lens injury stimulates adult mouse retinal ganglion cell axon regeneration via both macrophage- and lens-derived factors. Eur. J. Neurosci. 21, 2029-2034 (2005).
21. Yin, Y. et al. Macrophage-derived factors stimulate optic nerve regeneration. J. Neurosci. 23, 2284-2293 (2003).
22. Li, Y., Irwin, N., Yin, Y., Lanser, M. & Benowitz, L. I. Axon regeneration in goldfish and rat retinal ganglion cells: differential responsiveness to carbohydrates and cAMP. J. Neurosci. 23, 78307838 (2003).
23. Lorber, B., Berry, M., Logan, A. & Tonge, D. Effect of lens lesion on neurite outgrowth of retinal ganglion cells in vitro. Mol. Cell. Neurosci. 21, 301-311 (2002).
24. MacManus, J. P., Whitfield, J. F., Boynton, A. L., Durkin, J. P. & Swierenga, S. H. Oncomodulin—a widely distributed, tumour-specific, calcium-binding protein. Oncodev. Biol. Med. 3, 79-90 (1982).
25. Henzl, M. T., Larson, J. D. & Agah, S. Influence of monovalent cation identity on parvalbumin divalent ion-binding properties. Biochemistry 43, 2747-2763 (2004).
26. Henzl, M. T., Shibasaki, O., Comegys, T. H., Thalmann, I. & Thalmann, R. Oncomodulin is abundant in the organ of Corti. Hear. Res. 106, 105-111 (1997).
27. Meyer-Franke, A., Kaplan, M. R., Pfrieger, F. W. & Barres, B. A. Characterization of the signaling interactions that promote the survival and growth of developing retinal ganglion cells in culture. Neuron 15, 805-819 (1995).
28. Jo, S., Wang, E. & Benowitz, L. I. CNTF is an endogenous axon regeneration factor for mammalian retinal ganglion cells. Neuroscience 89, 579-591 (1999).
29. Mansour-Robaey, S., Clarke, D. B., Wang, Y. C., Bray, G. M. & Aguayo, A. J. Effects of ocular injury and administration of brain-derived neurotrophic factor on survival and regrowth of axotomized retinal ganglion cells. Proc. Natl. Acad. Sci. USA 91, 1632-1636 (1994).
30. Cui, Q., Yip, H. K., Zhao, R. C., So, K. F. & Harvey, A. R. Intraocular elevation of cyclic AMP potentiates ciliary neurotrophic factor-induced regeneration of adult rat retinal ganglion cell axons. Mol. Cell. Neurosci. 22, 4961 (2003).
31. Barres, B. A., Silverstein, B. E., Corey, D. P. & Chun, L. L. Immunological, morphological, and electrophysiological variation among retinal ganglion cells purified by panning. Neuron 1, 791-803 (1988).
32. Meyer-Franke, A. et al. Depolarization and cAMP elevation rapidly recruit TrkB to the plasma membrane of CNS neurons. Neuron 21, 681-693 (1998).
33. Maune, J. F., Beckingham, K., Martin, S. R. & Bayley, P. M. Circular dichroism studies on calcium binding to two series of Ca²⁺ binding site mutants of Drosophila melanogaster calmodulin. Biochemistry 31, 7779-7786 (1992).
34. Pauls, T. L., Cox, J. A. & Berchtold, M. W. The Ca²⁺-binding proteins parvalbumin and oncomodulin and their genes: new structural and functional findings. Biochim. Biophys. Acta 1306, 39-54 (1996).
35. Cai, D., Shen, Y., De Bellard, M., Tang, S. & Filbin, M. T. Prior exposure to neurotrophins blocks inhibition of axonal regeneration by MAG and myelin via a cAMP-dependent mechanism. Neuron 22, 89-101 (1999).
36. MacMicking, J., Xie, Q. W. & Nathan, C. Nitric oxide and macrophage function. Annu. Rev. Immunol. 15, 323-350 (1997).
37. Steinmetz, M. P. et al. Chronic enhancement of the intrinsic growth capacity of sensory neurons combined with the degradation of inhibitory proteoglycans allows functional regeneration of sensory axons through the dorsal root entry zone in the mammalian spinal cord. J. Neurosci. 25, 80668076 (2005).
38. Lu, X. & Richardson, P. M. Inflammation near the nerve cell body enhances axonal regeneration. J. Neurosci. 11, 972-978 (1991).
39. Rapalino, O. et al. Implantation of stimulated homologous macrophages results in partial recovery of paraplegic rats. Nat. Med. 4, 814821 (1998).
40. Porter, A. C. et al. MI muscarinic receptor signaling in mouse hippocampus and cortex. Brain Res. 944, 82-89 (2002).
41. Buxser, S., Decker, D. & Ruppel, P. Relationship among types of nerve growth factor receptors on PC12 cells. J. Biol. Chem. 265, 12701-12710 (1990).
42. Goldberg, J. L. et al. Retinal ganglion cells do not extend axons by default: promotion by neurotrophic signaling and electrical activity. Neuron 33, 689-702 (2002).
43. Streilein, J. W., Wilbanks, G. A., Taylor, A. & Cousins, S. Eye-derived cytokines and the immunosuppressive intraocular microenvironment: a review. Curr. Eye Res. 11. Suppl, 41-47 (1992).
44. Cohen-Cory, S. & Fraser, S. E. Effects of brain-derived neurotrophic factor on optic axon branching and remodelling in vivo. Nature 378, 192-196 (1995).
45. McKinnon, S. J. et al. Baculoviral IAP repeat-containing-4 protects optic nerve axons in a rat glaucoma model. Mol. Ther. 5, 780-787 (2002).
46. Otori, Y., Wei, J. Y. & Barnstable, C. J. Neurotoxic effects of low doses of glutamate on purified rat retinal ganglion cells. Invest. Opthalmol. Vis. Sci. 39, 972-981 (1998).

47. Hapak, R. C., Lammers, P. J., Palmisano, W. A., Birnbaum, E. R. & Henzl, M. T. Site-specific substitution of glutamate for aspartate at position 59 of rat oncomodulin. J. Biol. Chem. 264, 18751-18760 (1989).
48. Flanagan, J. G. et al. Alkaline phosphatase fusions of ligands or receptors as in situ probes for staining of cells, tissues, and embryos. Methods Enzymol. 327, 19-35 (2000).
49. Fu, K. et al. A potential approach for decreasing the burst effect of protein from PLGA microspheres. J. Pharm. Sci. 92, 1582-1591 (2003).
50. Gavazzi, I., Kumar, R. D., McMahon, S. B. & Cohen, J. Growth responses of different subpopulations of adult sensory neurons to neurotrophic factors in vitro. Eur. J. Neurosci. 11, 3405-3414 (1999).

Those skilled in the art will recognize, or be able to ascertain using no more than routine experimentation, many equivalents to the specific embodiments of the invention described herein. Such equivalents are intended to be encompassed by the following claims.

1. A method for treating and/or preventing peripheral nerve damage in a subject comprising administering to the subject a therapeutically effective amount of oncomodulin, to thereby treat and/or prevent peripheral nerve damage in the subject.

2. The method of claim 1 wherein the peripheral nerve damage is in the subject's spinal cord.

3. A method for treating and/or preventing spinal cord injury in a subject comprising administering to the subject a therapeutically effective amount of oncomodulin to thereby treat and/or prevent spinal cord injury in the subject.

4. The method of claim 1, further comprising the step of selecting a subject in need of treatment or prevention of peripheral nerve damage.

5. The method of claim 1, further comprising administering to said subject a cAMP modulator.

6. The method of claim 5, wherein said cAMP modulator is non-hydrolyzable cAMP analogues, forskolin, adenylate cyclase activators, macrophage-derived factors that stimulate cAMP, macrophage activators, calcium ionophores, membrane depolarization, phosphodiesterase inhibitors, specific phosphodiesterase IV inhibitors, beta2-adrenoreceptor inhibitors or vasoactive intestinal peptide.

7. The method of claim 1, further comprising administering mannose or a mannose derivative to said subject.

8. The method of claim 1, further comprising administering inosine to said subject.

9. The method of claim 5, wherein the cAMP modulator is forskolin.

10. The method of claim 1, wherein the peripheral nerve damage is the result of diabetic neuropathy.

11. The method of claim 1, wherein the peripheral nerve damage is the result of a viral or bacterial infection.

12. The method of claim 1, wherein the oncomodulin is administered topically.

13. The method of claim 1, wherein the oncomodulin is administered by local injection.

14. The method of claim 1, wherein the oncomodulin is administered to the subject in a pharmaceutically acceptable formulation.

15. The method of claim 1, wherein the subject is a mammal.

16. The method of claim 15, wherein the mammal is a human.

17. An article of manufacture comprising packaging material and a pharmaceutical agent contained within said packaging material, wherein said packaging material comprises a label which indicates said pharmaceutical may be administered, for a sufficient term at an effective dose, for treating and/or preventing peripheral nerve damage together with a pharmaceutically acceptable carrier, wherein the pharmaceutical agent comprises oncomodulin.

18. A pharmaceutical kit for the treatment and/or prevention of damage to peripheral nerves comprising the combination of:
(a) oncomodulin;
(b) an axogenic factor; and
(c) a cAMP modulator.

19. The kit of claim 18, wherein the axogenic factor is mannose, a mannose derivative or inosine.

20. The kit of claim 18, wherein the cAMP modulator is non-hydrolyzable cAMP analogues, forskolin, adenylate cyclase activators, macrophage-derived factors that stimulate cAMP, macrophage activators, calcium ionophores, membrane depolarization, phosphodiesterase inhibitors, specific phosphodiesterase IV inhibitors, beta2-adrenoreceptor inhibitors or vasoactive intestinal peptide.

21. (canceled)

22. A method for inhibiting the axogenic effects of oncomodulin on a neuron comprising contacting an inhibitor of oncomodulin to the neuron.

23. The method of claim 22 wherein the neuron is in a subject in need of inhibition of oncomodulin axogenic effects, and contacting is achieved by administering the inhibitor to the subject.


SEQUENCE LISTING (3 sequences)

SEQ ID NO: 1 - protein, 14 aa, Artificial Sequence
  Description of Artificial Sequence Synthetic peptide
KSLMDAADNDGDGK

SEQ ID NO: 2 - DNA, 21 bp, Artificial Sequence
  Description of Artificial Sequence Synthetic primer
atgagcatcacggacatcctg

SEQ ID NO: 3 - DNA, 18 bp, Artificial Sequence
  Description of Artificial Sequence Synthetic primer
gtcctttaccacgtgaga